(19) United States
(12) Patent Application Publication
(10) Pub. No.: US 2011/0264185 A1
(43) Pub. Date: Oct. 27, 2011

(54) BALLOON CATHETER AND BALLOON MOLD TO FACILITATE MARKER PLACEMENT AND METHOD FOR USING SAME

(76) Inventor: Thomas Haslinger, Sun City, CA (US)

(21) Appl. No.: 12/764,754

(22) Filed: Apr. 21, 2010

(51) Int. Cl. A61F 2/84; B29C 33/12; B28B 11/08; A61M 25/10

(52) U.S. Cl. 623/1.11; 604/103.08; 425/169; 264/293

(57) ABSTRACT

A catheter balloon is formed from a mold process with a circumferential demarcation is formed on the outer surface using a groove or ridge on the mold surface. The demarcation is located at the transition between the body portion of the catheter balloon and the neck or taper section. The presence of the demarcation serves to identify the transition between the two regions for placing and positioning a visual marker inside the balloon.

### BACKGROUND

This invention generally relates to intravascular balloon catheters such as those used in percutaneous transluminal coronary angioplasty (PTCA) and stent delivery, and more particularly to a catheter balloon and mold for creating a balloon that permits reliable securement of positioning markers and stents.

PTCA is a widely used procedure for the treatment of coronary heart disease. In this procedure, a balloon dilatation catheter is advanced into the patient's coronary artery and the balloon on the catheter is inflated within the stenotic region of the patient's artery to open up the arterial passageway and thereby increase the blood flow there through. To facilitate the advancement of the dilatation catheter into the patient's coronary artery, a guiding catheter having a preshaped distal tip is first percutaneously introduced into the cardiovascular system of a patient by the Seldinger technique or other method through the brachial or femoral arteries.

The catheter is advanced until the preshaped distal tip of the guiding catheter is disposed within the aorta adjacent the ostium of the desired coronary artery, and the distal tip of the guiding catheter is then maneuvered into the ostium. A balloon dilatation catheter may then be advanced through the guiding catheter into the patient's coronary artery over a guidewire until the balloon on the catheter is disposed within the stenotic region of the patient's artery. The balloon is inflated to open up the arterial passageway and increase the blood flow through the artery. Generally, the inflated diameter of the balloon is approximately the same diameter as the native diameter of the body lumen being dilated so as to complete the dilatation but not over expand the artery wall. After the balloon is finally deflated, blood flow resumes through the dilated artery and the dilatation catheter can be removed.

In a large number of angioplasty procedures, there may be a restenosis, i.e. reformation of the arterial plaque. To reduce the restenosis rate and to strengthen the dilated area, physicians may implant an intravascular prosthesis or “stent” inside the artery at the site of the lesion. Stents may also be used to repair vessels having an intimal flap or dissection or to generally strengthen a weakened section of a vessel. Stents are usually delivered to a desired location within a coronary artery in a contracted condition on a balloon of a catheter which is similar in many respects to a balloon angioplasty catheter, and expanded to a larger diameter by expansion of the balloon. The balloon is then deflated to remove the catheter and the stent is left in place within the artery at the site of the dilated lesion.

To accurately place the balloon at the desired location, visual markers on the inner member are utilized that are read by machines outside the body. For example, in the case where a balloon catheter is used with an fluoroscope, the radiopaque marker may be observed visually on a screen while the procedure is taking place. In many cases, the markers must be precisely located to ensure accurate placement of the balloon in the affected area. When stents are being deployed the location of the beginning and ending point of the stent can be crucial to the success of the procedure. In such cases, it is preferred that the markers be located very specifically near the junction of the body portion of the balloon with the tapered portion of the balloon. However, it is also important that the marker not be located too far inside the tapered portion of the balloon. Unfortunately, the manufacturing process does not readily lend itself to a precise determination as to where to apply the marker such that it is at the extreme end of the working portion of the balloon but does not extend too far into the tapered portion.

### SUMMARY OF THE INVENTION

The present invention addresses the problem above by using a modified mold to create a groove, ring, or visual discontinuity on the balloon that facilitates the proper positioning of radiopaque markers in relation to a balloon. In the case of the circumferential groove or raised ring, the visual discontinuity can be created by modifying an ordinary balloon mold to have an embossed complimentary ring or groove at a precise location at or in close vicinity to the balloon's shoulders. This raised or recessed line around the entire circumference of the balloon can easily identify the desired location of the visual markers. The raised or recessed ring(s) will enable the manufacturing personnel to locate and position the visual markers precisely according to the respective manufacturing process instructions. This also aids in the placement and retention of stents that are mounted on the balloon in procedures that use this feature.

### BRIEF DESCRIPTION OF THE DRAWINGS

FIG. 1 is an elevated view partially in section of a balloon catheter of the present invention;

FIG. 2 is a transverse cross sectional view of the balloon catheter of FIG. 1 taken along lines 2-2;

FIG. 3 is a transverse cross sectional view of the balloon catheter of FIG. 1 taken along lines 3-3;

FIG. 4 is an enlarged view of the balloon catheter of FIG. 1 with a vascular stent mounted thereon;

FIG. 5 is an enlarged view of the stent of FIG. 4 disposed in a patient's vascular after removal of the balloon;

FIG. 6. is an even more enlarged view of the distal end of the balloon and stent of FIG. 4 showing the molded circumferential ring;

FIG. 7 is a cut-away view of a mold for forming the balloon of the present invention and a balloon tubing prior to forming;

FIG. 8 is a cut-away view of the mold of FIG. 7 after pressurization and heating to form the balloon of the present invention;

FIG. 9 is an enlarged view of another embodiment of the present invention with a vascular stent mounted thereon;

FIG. 10 is an even more enlarged view of the distal end of the balloon and stent of FIG. 4 showing the molded circumferential groove;

FIG. 11 is a cut-away view of a mold for forming the balloon of FIGS. 9 and 10 prior to forming; and

FIG. 12 is a cut-away view of the mold of FIG. 11 after pressurization and heating to form the balloon of FIGS. 9 and 10.

### DETAILED DESCRIPTION OF THE PREFERRED EMBODIMENTS

FIG. 1 shows a balloon catheter that can be used to illustrate the features of the invention. The catheter 10 of the invention generally comprises an elongated catheter shaft 11 having a proximal section 12, a distal section 13, an inflatable balloon 14 on the distal section 13 of the catheter shaft 11, and an adapter 17 mounted on the proximal section 12 of shaft 11. In FIG. 1, the catheter 10 is illustrated within a greatly enlarged view of a patient's body lumen 18, prior to expansion of the balloon 14, adjacent the tissue to be injected with therapeutic agents.

In the embodiment illustrated in FIG. 1, the catheter shaft 11 has an outer tubular member 19 and an inner tubular member 20 disposed within the outer tubular member and defining, with the outer tubular member, inflation lumen 21. Inflation lumen 21 is in fluid communication with the interior chamber 15 of the inflatable balloon 14. The inner tubular member 20 has an inner lumen 22 extending therein which is configured to slidably receive a guidewire 23 suitable for advancement through a patient's coronary arteries. The distal extremity of the inflatable balloon 14 is sealingly secured to the distal extremity of the inner tubular member 20 and the proximal extremity of the balloon is sealingly secured to the distal extremity of the outer tubular member 19.

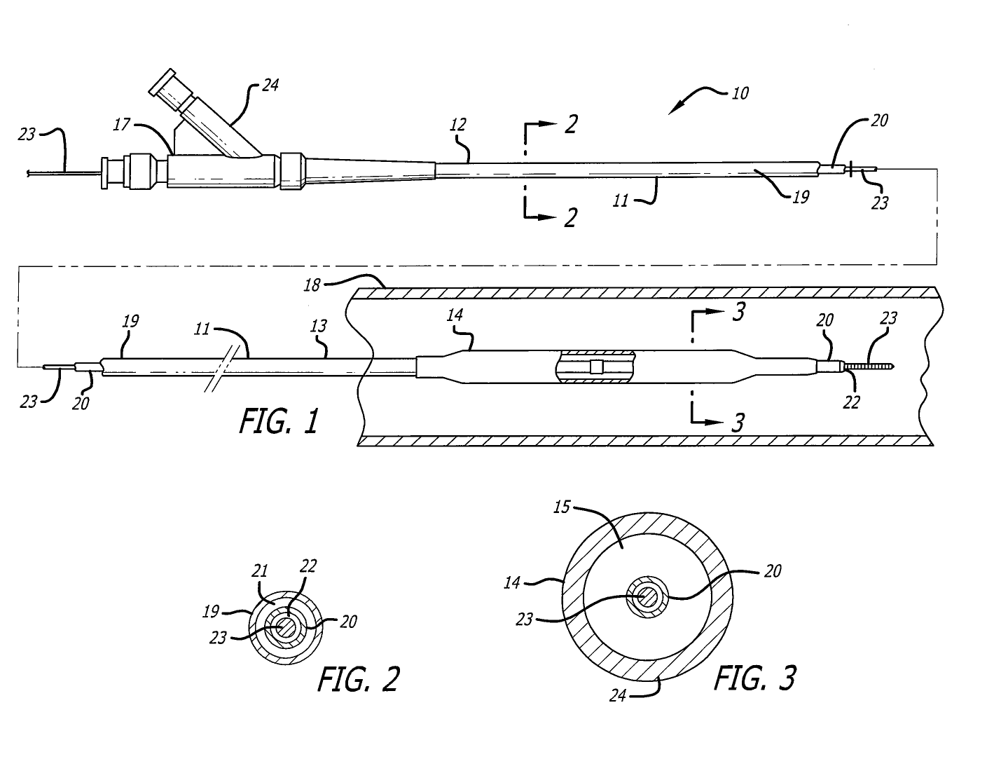

FIGS. 2 and 3 show transverse cross sections of the catheter shaft 11 and balloon 14, respectively, illustrating the guidewire receiving lumen 22 of the guidewire's inner tubular member 20 and inflation lumen 21 leading to the balloon interior 15. The balloon 14 can be inflated by a fluid such as air, saline, or other fluid that is introduced at the port in the side arm 24 into inflation lumen 21 contained in the catheter shaft 11, or by other means, such as from a passageway formed between the outside of the catheter shaft 11 and the member forming the balloon 14, depending on the particular design of the catheter. The details and mechanics of the mode of inflating the balloon vary according to the specific design of the catheter, and are omitted from the present discussion.

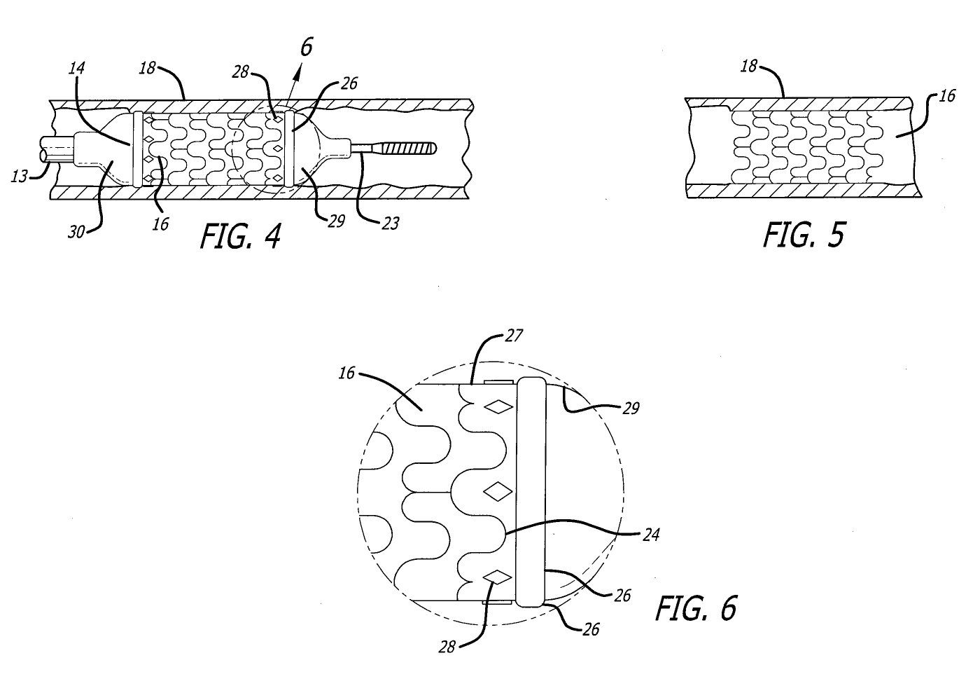

FIG. 4 illustrates an embodiment of the catheter of FIG. 1 with a vascular stent 16 mounted thereon. The stent 16 can be made in many ways. One method of making the stent is to cut a thin-walled tubular member, such as stainless steel tubing to remove portions of the tubing in the desired pattern for the stent, leaving relatively untouched the portions of the metallic tubing which are to form the stent. The stent also can be made from other metal alloys such as tantalum, nickel-titanium, cobalt-chromium, titanium, shape memory and superelastic alloys, and the Nobel metals such as gold or platinum. It is preferred to cut the tubing in the desired pattern by means of a machine-controlled laser as is well known in the art. Stents function to hold open a segment of a blood vessel or other body lumen such as a renal or coronary artery. At present, there are numerous commercial stents being marketed throughout the world. While some of these stents are flexible and have the appropriate radial rigidity needed to hold open a vessel or artery, there typically is a tradeoff between flexibility and radial strength and the ability to tightly compress or crimp the stent onto a catheter so that it does not move relative to the catheter or dislodge prematurely prior to controlled implantation in a vessel. Currently, to secure a stent 16 on a balloon 14, after the stent is crimped onto the deflated balloon such that the balloon partially protrudes through the stent struts. During this process, the balloon and stent are placed in a heated mold and pressurized. The balloon protrusions then acts as holds to secure the stent in place.

In a typical procedure to implant stent 16, the guide wire 23 is advanced through the patient's vascular system by well known methods so that the distal end of the guide wire is advanced past the location for the placement of the stent in the body lumen 18. Prior to implanting the stent 16, the cardiologist may wish to perform an angioplasty procedure or other procedure (i.e., atherectomy) in order to open the vessel and remodel the diseased area. Thereafter, the stent delivery catheter assembly 10 is advanced over the guide wire 23 so that the stent 16 is positioned in the target area. The balloon 14 is inflated so that it expands radially outwardly and in turn expands the stent 16 radially outwardly until the stent 16 bears against the vessel wall of the body lumen 18. The balloon 14 is then deflated and the catheter withdrawn from the patient's vascular system, leaving the stent 16 in place to dilate the body lumen. The guide wire 23 typically is left in the lumen for post-dilatation procedures, if any, and subsequently is withdrawn from the patient's vascular system. As depicted in FIG. 4, the balloon 14 is fully inflated with the stent 16 expanded and pressed against the vessel wall, and in FIG. 5, the implanted stent 16 remains in the vessel after the balloon has been deflated and the catheter assembly and guide wire have been withdrawn from the patient.

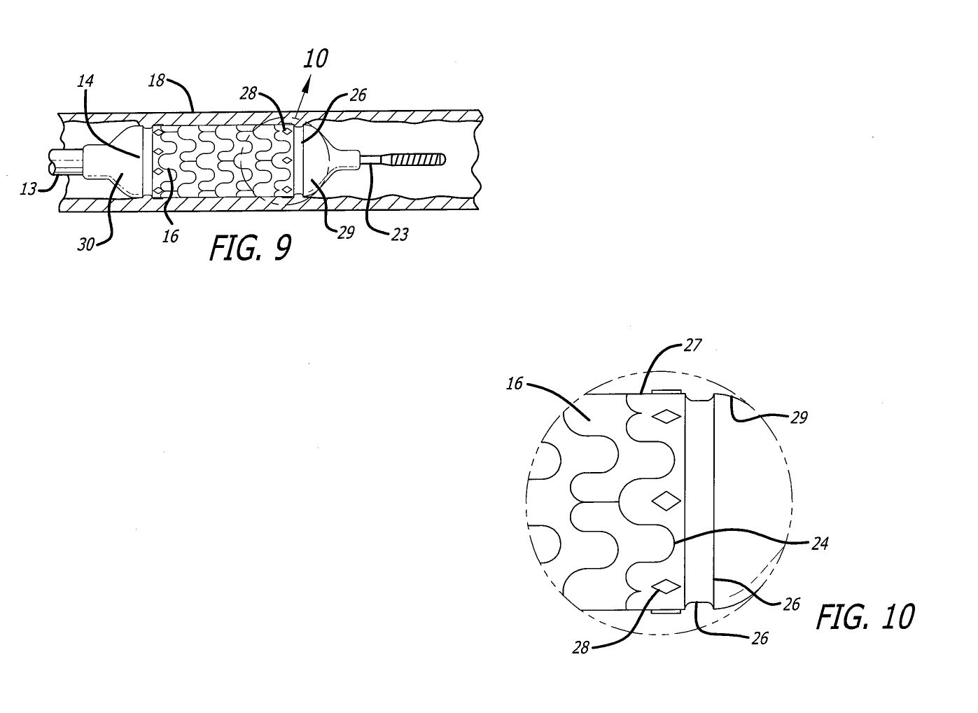

FIG. 6 illustrates a close up section of the balloon 14 showing the circumferential raised ring 26 at the juncture of the body section 27 of the balloon 14 and the onset of the tapered section 29. The raised ring 26 provides a visual aid of the end of the body or working portion 27 of the balloon, and consequently the location of the edge 24 of the stent 16. For example, in a first preferred embodiment the raised ring 26 is located one millimeter from the beginning of the taper section. Radiopaque markers 28 on the inner member 20 can be aligned with the ring 26 and used to locate both the balloon 14 and the stent 16. It is to be understood that a similar ring or groove 26 will ordinarily be formed at the proximal end of the working section 27 of the balloon 14 where it tapers into the proximal taper portion 30. The markers 28 are observed under the fluoroscope and can be used to precisely locate the catheter, the balloon 14, and the stent 16. FIGS. 9 and 10 illustrate a second embodiment where the raised ring is replaced with a recessed groove 26.

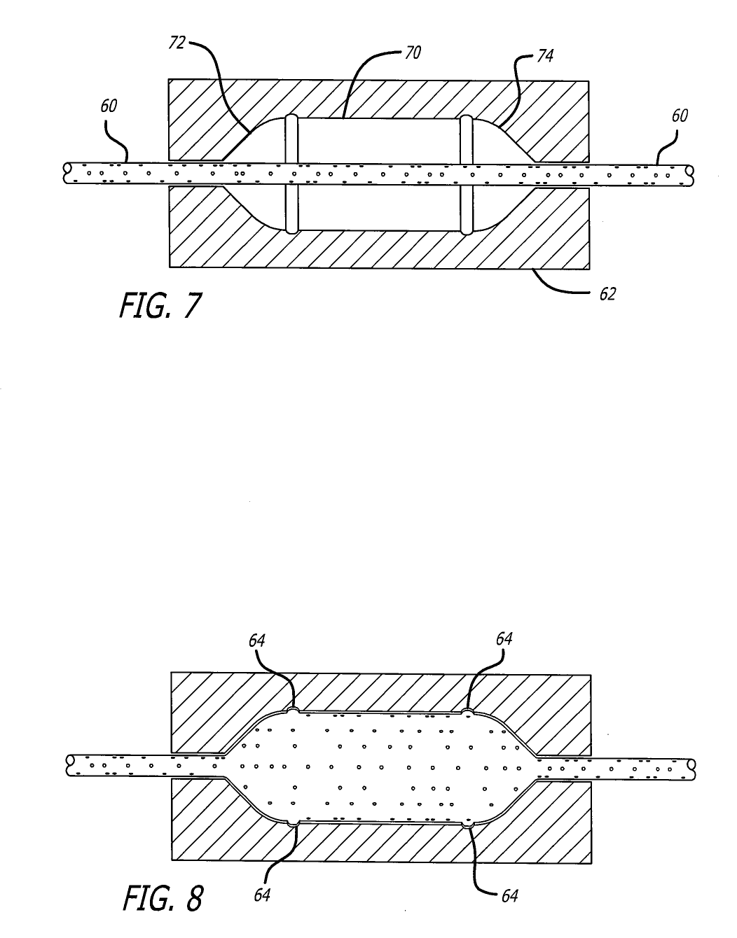

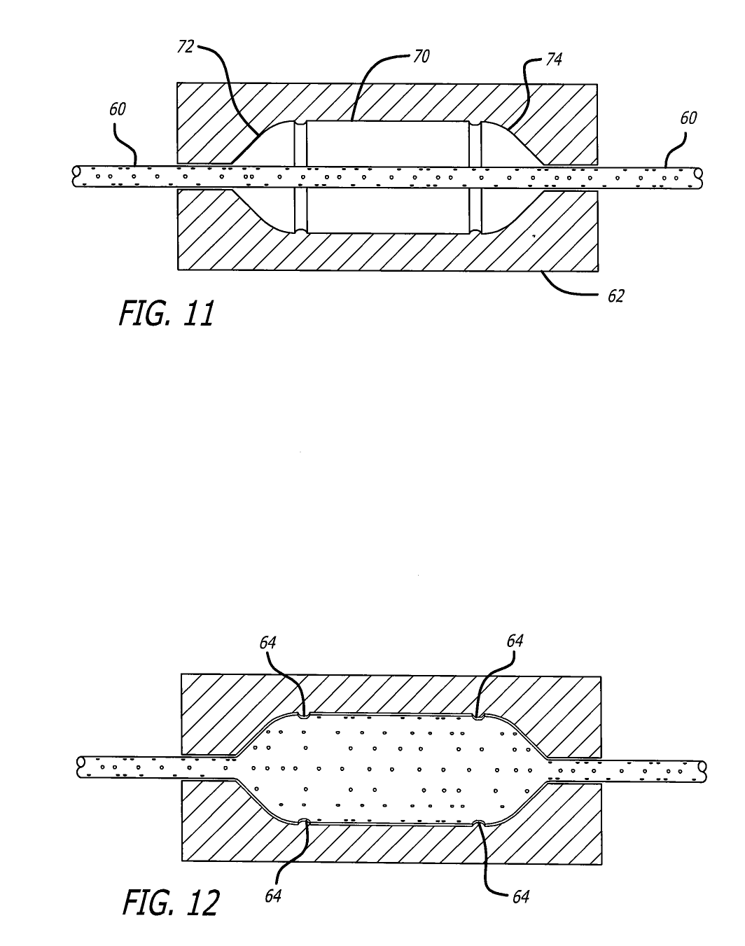

The balloon 14 is formed using conventional balloon technologies, such as blow molding as illustrated in FIGS. 7 and 8. A tube 60 of balloon material is inserted into a mold 62 having the desired balloon shape. The mold 62 has a constant radius wall 70 and an increasing radial section 72 at a first end and a decreasing radial section 74 at a second end, and further includes a circumferential groove or ridge 64 on the constant radius wall section at the transition to the radially increasing and decreasing portions. The groove 64 fills with balloon material as the tube is expanded and heated to form a balloon with the desired raised ring 26 at the edges of the working section of the balloon 14. Alternatively, the ridge 64 forms a groove 26 in the balloon 14 at the edges of the working section of the balloon 14. The balloon material is maintained in the heated and pressurized state until the balloon is formed to cause the tubing 60 to expand to the final shape within the mold 62, including the formation of the rings/grooves at the desired locations. This will result in a balloon that includes the rings or grooves 26 shown in FIGS. 4 and 6. FIGS. 11 and 12 correspond to FIGS. 7 and 8 where the raised ring has been replaced with the recessed groove.

While particular forms of the invention have been illustrated and described, it will be apparent to those skilled in the art that various modifications can be made without departing from the spirit and scope of the invention. Accordingly, it is not intended that the invention be limited except by the appended claims.

What is claimed:

1. A catheter balloon, comprising:
a working body section and a proximal and a distal taper section; and
a raised ring encircling the balloon and demarking the transition between the working body section and an adjacent taper section.

2. The catheter balloon of claim 1, further comprising a second raised ring encircling the balloon and demarking the transition between the working body and another adjacent taper section.

3. The catheter balloon of claim 1, further comprising a stent mounted on the balloon, wherein the raised ring locates a first end of the stent on the balloon.

4. The catheter balloon of claim 1, wherein the balloon is formed by blow molding.

5. A catheter balloon, comprising:
a working body section and a proximal and distal taper section; and
a recessed groove encircling the balloon and demarking the transition between the working body section and an adjacent taper section.

6. The catheter balloon of claim 5, further comprising a second recessed groove encircling the balloon and demarking the transition between the working body and another adjacent taper section.

7. The catheter balloon of claim 5, further comprising a stent mounted on the balloon, wherein the recessed groove locates a first end of the stent on the balloon.

8. The catheter balloon of claim 5, wherein the balloon is formed by blow molding.

9. A mold for a catheter balloon, comprising:
a first inner wall defining a constant radius void for forming a working portion of the balloon, second and third inner walls defining first and second neck portions of the balloon, and an inwardly projecting circumferential ridge located at a transition between the first wall and one of the second and third walls to form a circumferential groove on an outer surface of the balloon on the working portion adjacent the one of the second and third walls.

10. The mold of claim 9, further comprising a second inwardly projecting circumferential ridge located at a transition between the first wall and another of the second and third walls to form a circumferential groove on the outer surface of the balloon on the working portion adjacent the another one of the second and third walls.

11. A method for locating a positioning marker on a catheter balloon, comprising:
providing a mold for a balloon having an inner wall defining a shape of a balloon;
positioning an indention on the mold to create a circumferential demarcation on an outer surface of the balloon where a location of the indentation coincides with a desired location of the positioning marker; and
incorporating a positioning marker in the balloon using the demarcation on the outer surface to place the positioning marker at the desired location.
